(19) United States
(12) Patent Application Publication
(10) Pub. No.: US 2020/0215288 A1
(43) Pub. Date: Jul. 9, 2020

(54) Nasal continuous positive airway pressure device and system

(71) Applicant: INNO3 APS, Rønde (DK)

(72) Inventors: Ann-Christina Lykke Rasmussen, Ronde (DK); Michael Motzfeldt, Ronde (DK)

(21) Appl. No.: 16/628,305

(22) PCT Filed: Jun. 28, 2018
(86) PCT No.: PCT/EP2018/067430

§ 371 Date: Jan. 3, 2020

(30) Foreign Application Priority Data
Jul. 3, 2017 (DK) PA 2017 00397

(51) Int. Cl. A61M 16/06; A61M 16/08

(52) U.S. Cl. CPC A61M 16/0672; A61M 16/0858; A61M 2205/42; A61M 2202/0208; A61M 16/0875

(57) ABSTRACT

A nasal continuous positive airway pressure (CPAP) device 100 includes a neck region 200 connected to a mask or nasal prong; and an elongated flexible tube 300. The elongated flexible tube 300 comprises co-axial inner 310 and outer 320 tubes. The inner 310 and outer 320 tubes are spaced to define a first lumen 330 within the outer tube 320. A second lumen 340 is defined by the inner tube wall alone. The second lumen 340 is configured to be connected to a breathable gas supply such as air or oxygen at the distal end 304 of the elongated flexible tube 300, and to the neck region 200 at the proximal end 302 of the elongated flexible tube 300. The first lumen 330 is configured to be connected to the neck region 200 at the proximal end 302 of the elongated flexible tube 300.

### TECHNICAL FIELD OF THE INVENTION

The present invention relates to the field of continuous positive airway pressure (CPAP) therapy, more specifically to nasal continuous positive airway pressure (nCPAP) systems.

### BACKGROUND OF THE INVENTION

Continuous positive airway pressure (CPAP) therapy has been employed for many years to treat patients experiencing respiratory difficulties and/or insufficiencies. More recently, CPAP therapy has been advanced as being useful in assisting patients with under-developed lungs (especially infants, such as neonates), by preventing lung collapse during exhalation and assisting lung expansion during inhalation.

In general terms, CPAP therapy entails the continuous transmission of positive pressure into the lungs of a spontaneously breathing patient throughout the respiratory cycle. CPAP can be delivered to the patient using a variety of patient interface devices, for example an endotracheal tube. With infants, however, it is more desirable to employ a less invasive patient interface device, in particular one that interfaces directly or indirectly with the nasal airways via the patient's nares (e.g., mask or nasal prongs). Such systems are commonly referred to as nasal continuous positive airway pressure (nCPAP) systems.

In theory, the CPAP system should deliver a constant, stable pressure to the patient's airways. With conventional, ventilator-based CPAP devices, a relative constant and continuous flow of gas (e.g., air, oxygen gas, etc.) is delivered into the patient's airways, with this airflow creating a pressure within the patient's lungs via a restriction placed on outflow from the patient. Unfortunately, this continuous flow can have an adverse effect on the patient's respiratory synchrony. More particularly, the patient is required to exhale against the incoming gas, thus increasing the patient's work of breathing. For an infant with under developed lungs, even a slight increase in the required work of breathing may render the CPAP system in question impractical.

More recently, nCPAP systems have been developed that incorporate a variable flow concept in combination with separate channels for inspiratory and expiratory gas to and from the patient. When the patient inhales, the incoming gas takes the path of least resistance and is directed to the patient's airways. Upon expiration, the gas again takes the path of least resistance and goes out an exhalation or exhaust tube, thus reducing resistance during the expiratory phase.

High levels of noise are a stressful factor that can have tremendous effects on a neonate's recovery. While highly specialized treatments can greatly improve a neonate's medical condition, physical surroundings are rarely regarded as influential in his or her care. The crowded neonatal intensive care units are usually packed with nurses, equipment and lots of noise that affect the neonates \check{ } stress levels. These effects are clearly noticeable in neonates \check{ } monitors that show how a lot of noise can agitate their fragile hearts. In addition, these loud, crowded environments are also not particularly family friendly, and parents often do not feel comfortable about spending time there (Praskach, Ana, “Nature, daylight and sound: A sensible environment for the families, staff and patients of neonatal intensive care units” (2009). Graduate Theses and Dissertations). A general problem with current nCPAP systems is that noise is generated close to the head of the patient.

### OBJECT OF THE INVENTION

The objective of the present invention is to provide an nCPAP system with a reduced noise generation close to the head of the patient.

### DESCRIPTION OF THE INVENTION

A first aspect of the present invention relates to a nasal continuous positive airway pressure device comprising:
a neck region a) adapted for being connected to a mask or nasal prong, or b) connected to a mask or nasal prong;
a gas inlet tube with a proximal end adapted for being connected to the mask or nasal prong; and
an exhaust tube with a proximal end adapted for being connected to the mask or nasal prong;
   wherein a) the gas inlet tube is positioned within the exhaust tube, or b) the exhaust tube is positioned within the gas inlet tube.

A second aspect of the present invention relates to a nasal continuous positive airway pressure device comprising:
a neck region a) adapted for being connected to a mask or nasal prong, or b) connected to a mask or nasal prong;
an elongated flexible tube comprising co-axial inner and outer tubes, and
   wherein the inner and outer tubes are spaced to define a first lumen within the outer tube, and wherein a second lumen is defined by the inner tube wall alone; wherein a) the second lumen is configured to be connected to an oxygen gas supply at the distal end of the elongated flexible tube, and to the neck region at the proximal end of the elongated flexible tube; and wherein the first lumen is configured to be connected to the neck region at the proximal end of the elongated flexible tube; or b) the first lumen is configured to be connected to an oxygen gas supply a the distal end of the elongated flexible tube, and to the neck region at the proximal end of the elongated flexible tube; and wherein the second lumen is configured to be connected to the neck region at the proximal end of the elongated flexible tube. This configuration allows for a very compact nasal continuous positive airway pressure device that appears less dangerous to the parents of the neonate. More importantly, this configuration reduces the stress of the neonate by removing the exhaust air and noise from the neonate face.

In one or more embodiments, the second lumen is configured to be connected to an oxygen gas supply a the distal end of the elongated flexible tube, and to the neck region at the proximal end of the elongated flexible tube; wherein the first lumen is configured to be connected to the neck region at the proximal end of the elongated flexible tube; and wherein the distal end of the inner tube extends out of the outer tube at its distal end, such that the first lumen is in fluid communication with the surrounding atmosphere at the distal end of the outer tube. Preferably, the length of the outer tube is at least 20 cm, such as within the range of 20-200 cm, e.g. within the range of 25-150 cm, such as within the range of 30-125 cm, e.g. within the range of 35-100 cm, such as within the range of 40-90 cm, e.g. within the range of 45-80 cm, such as within the range of 50-70 cm, e.g. within the range of 55-65 cm. This configuration reduces the stress of the neonate by removing the exhaust air and noise from the neonates face.

In one or more embodiments, the second lumen is configured to be connected to an oxygen gas supply a the distal end of the elongated flexible tube, and to the neck region at the proximal end of the elongated flexible tube; wherein the first lumen is configured to be connected to the neck region at the proximal end of the elongated flexible tube; wherein the neck region comprises:
a tube part adapted for being connected to the proximal end of the inner tube, and comprising a lumen in fluid communication with the second lumen; and
a cone part adapted for being connected to the proximal end of the outer tube, and comprising a lumen in fluid communication with the first lumen; wherein the cone part is positioned around at least a part of the tube part;
   wherein the lumen of the tube part is in fluid communication with the lumen of the cone part through one or more apertures in the wall of the tube part. This configuration decreases the patient's work of breathing during exhalation.

In one or more embodiments, the neck region comprises a gas pressure port adapted for being connected to means for measuring the gas pressure within the neck region. This configuration allows for control of the oxygen supply to the patient.

In one or more embodiments, the tube part comprises a gas pressure port adapted for being connected to means for measuring the gas pressure within the tube part.

In one or more embodiments, the tube part comprises a straight tube section, and a bend tube section.

In one or more embodiments, the tube part comprises a straight tube section, and a bend tube section, and wherein the one or more apertures are positioned in the straight tube section.

In one or more embodiments, the tube part comprises a straight tube section, and a bend tube section, and wherein the one or more apertures are positioned in the straight tube section, and wherein the gas pressure port is positioned in the bend tube section.

In one or more embodiments, the one or more apertures are configured as an elongate recess.

In one or more embodiments, one or more apertures are circular.

In one or more embodiments, lumen of the tube part is in fluid communication with the lumen of the cone part through a plurality of apertures in the wall of the tube part.

As used in the specification and the appended claims, the singular forms “a”, “an”, and “the” include plural referents unless the context clearly dictates otherwise. Ranges may be expressed herein as from “about” or “approximately” one particular value and/or to “about” or “approximately” another particular value.

When such a range is expressed, another embodiment includes from the one particular value and/or to the other particular value. Similarly, when values are expressed as approximations, by use of the antecedent “about”, it will be understood that the particular value forms another embodiment.

It should be noted that embodiments and features described in the context of one of the aspects of the present invention also apply to the other aspects of the invention.

### BRIEF DESCRIPTION OF THE FIGURES

FIG. 1 is a perspective view of a nasal continuous positive airway pressure device in accordance with various embodiments of the invention;

FIG. 2 is a top view of a nasal continuous positive airway pressure device in accordance with various embodiments of the invention;

FIG. 3 is the cross-sectional view A-A of FIG. 2, shown as a side view; and

FIGS. 4-5 are a detailed view B of FIG. 3.

### REFERENCES

100 Nasal continuous positive airway pressure device
200 Neck region
210 Tube part
212 Aperture
214 Lumen
216 Gas pressure port
218 Straight tube section
219 Bend tube section
220 Cone part
222 Lumen
300 Flexible tube
302 Proximal end
304 Distal end
310 Inner tube
312 Distal end
320 Outer tube
322 Distal end
330 First lumen
340 Second lumen

### DETAILED DESCRIPTION OF THE INVENTION

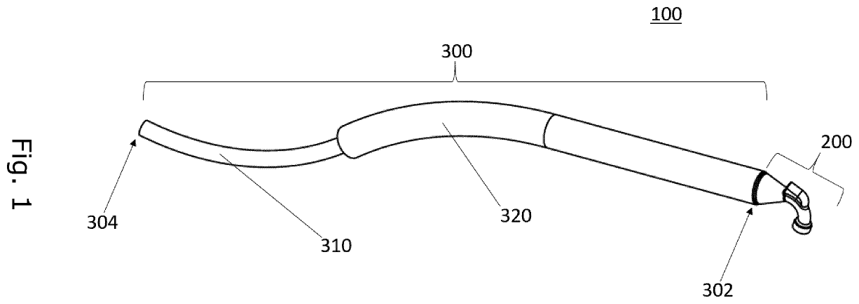

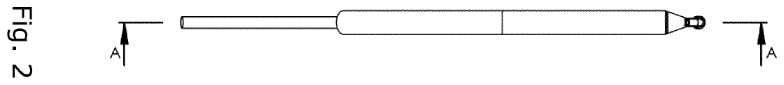

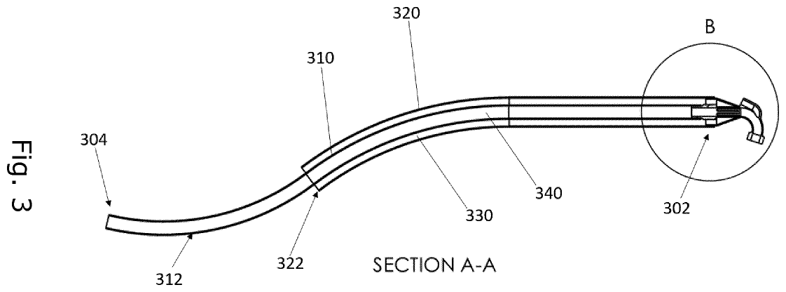

FIG. 1 is a perspective view of a nasal continuous positive airway pressure device in accordance with various embodiments of the invention. The nasal continuous positive airway pressure device 100 comprises a neck region 200 adapted for being connected to a mask or nasal prong (not shown); and an elongated flexible tube 300. The elongated flexible tube 300 comprises co-axial inner 310 and outer 320 tubes. The inner 310 and outer 320 tubes are spaced to define a first lumen 330 within the outer tube 320 (FIG. 3). A second lumen 340 is defined by the inner tube wall alone. The second lumen 340 is configured to be connected to an oxygen gas supply at the distal end 304 of the elongated flexible tube 300, and to the neck region 200 at the proximal end 302 of the elongated flexible tube 300. The first lumen 330 is configured to be connected to the neck region 200 at the proximal end 302 of the elongated flexible tube 300.

The distal end 312 of the inner tube 310 extends out of the outer tube 320 at its distal end 322, such that the first lumen 330 is in fluid communication with the surrounding atmosphere at the distal end 322 of the outer tube 320 tube.

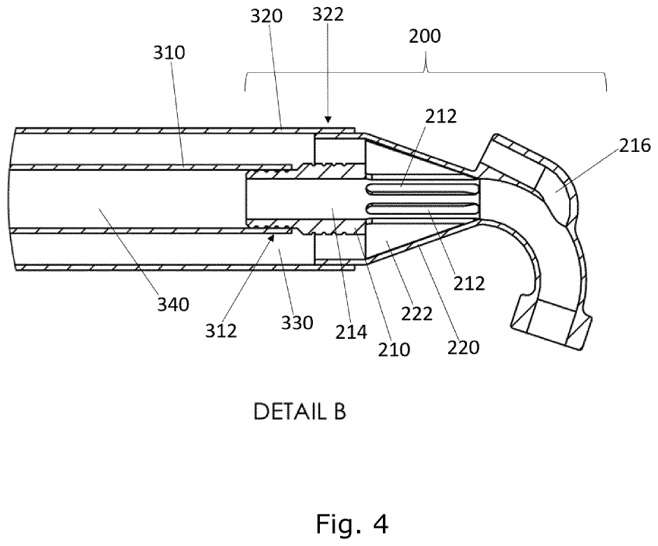

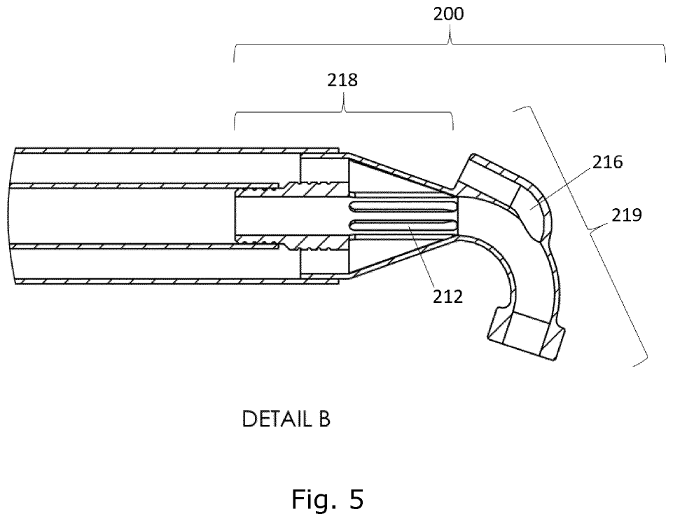

FIGS. 4-5 are a detailed view B of FIG. 3. The neck region 200 comprises a tube part 210 adapted for being connected to the proximal end 312 of the inner tube 310 and comprising a lumen 214 in fluid communication with the second lumen 340. The neck region 200 also comprises a cone part 220 adapted for being connected to the proximal end 322 of the outer tube 320 and comprising a lumen 222 in fluid communication with the first lumen 330. The cone part 220 is positioned around a part of the tube part 210. The lumen 214 of the tube part 210 is in fluid communication with the lumen 222 of the cone part 220 through a plurality of apertures 212 in the wall of the tube part 210.

The tube part 210 comprises a straight tube section 218, and a bend tube section 219. A plurality of apertures 212 are positioned in the straight tube section 218. Furthermore, a gas pressure port 216 is positioned in the bend tube section 219.

1. A nasal continuous positive airway pressure device (100) comprising:
a neck region (200) a) adapted for connection to a mask or nasal prong, or b) connection to a mask or nasal prong; and
an elongated flexible tube (300) comprising co-axial inner (310) and outer (320) tubes, and wherein the inner (310) and outer (320) tubes are spaced to define a first lumen (330) within the outer tube (320), and wherein a second lumen (340) is defined by an inner tube wall alone; wherein a) the second lumen (340) is configured for connection to an oxygen gas supply at the distal end (304) of the elongated flexible tube (300), and to the neck region (200) at the proximal end (302) of the elongated flexible tube (300); and wherein the first lumen (330) is configured for connection to the neck region (200) at the proximal end (302) of the elongated flexible tube (300); or b) the first lumen (330) is configured for connection to an oxygen gas supply a the distal end (304) of the elongated flexible tube (300), and to the neck region (200) at the proximal end (302) of the elongated flexible tube (300); and wherein the second lumen (340) is configured for connection to the neck region (200) at the proximal end (302) of the elongated flexible tube (300);
characterized in that the second lumen (340) is configured for connection to an oxygen gas supply a the distal end (304) of the elongated flexible tube (300), and to the neck region (200) at the proximal end (302) of the elongated flexible tube (300); wherein the first lumen (330) is configured for connection to the neck region (200) at the proximal end (302) of the elongated flexible tube (300); wherein the neck region (200) comprises:
a tube part (210) adapted for connection to the proximal end (312) of the inner tube (310), and comprising a lumen (214) in fluid communication with the second lumen (340); and
a cone part (220) adapted for connection to the proximal end (322) of the outer tube (320), and comprising a lumen (222) in fluid communication with the first lumen (330); wherein the cone part (220) is positioned around at least a part of the tube part (210);
wherein the lumen (214) of the tube part (210) is in fluid communication with the lumen (222) of the cone part (220) through one or more apertures (212) in the wall of the tube part (210).

2. The nasal continuous positive airway pressure device (100) according to claim 1, characterized in that the neck region (200) comprises a gas pressure port adapted for connection to means for measuring the gas pressure within the neck region (200).

3. The nasal continuous positive airway pressure device (100) according to claim 2, characterized in that the tube part (210) comprises a gas pressure port (216) adapted for connection to means for measuring the gas pressure within the tube part (210).

4. The nasal continuous positive airway pressure device (100) according to claim 3, characterized in that the tube part (210) comprises a straight tube section (218), and a bend tube section (219), and wherein the one or more apertures (212) are positioned in the straight tube section (218), and wherein the gas pressure port (216) is positioned in the bend tube section (219).

5. The nasal continuous positive airway pressure device (100) according to claim 1, characterized in that the one or more apertures (212) are configured as an elongate recess.

6. The nasal continuous positive airway pressure device (100) according to claim 1, characterized in that the one or more apertures (212) are circular.

7. The nasal continuous positive airway pressure device (100) according to claim 1, characterized in that the lumen (214) of the tube part (210) is in fluid communication with the lumen (222) of the cone part (220) through a plurality of apertures (212) in the wall of the tube part (210).

8. The nasal continuous positive airway pressure device (100) according to claim 1, characterized in that the outer tube (320) is at least 20 cm.
